(19) United States
(12) Patent Application Publication
(10) Pub. No.: US 2019/0241853 A1
(43) Pub. Date: Aug. 8, 2019

(54) COMPOSITIONS COMPRISING MICROALGAE AND METHODS OF PRODUCING AND USING SAME

(71) Applicant: MICRO GREEN TECHNOLOGIES LTD., RaAnana (IL)

(72) Inventors: Vladimir GOLTSMAN, Petach-Tikva (IL); Aharon COHEN, Ramot-HaShavim (IL)

(73) Assignee: Micro Green Technologies Ltd., RaAnana (IL)

(21) Appl. No.: 16/316,682

(22) PCT Filed: Jul. 12, 2017
(86) PCT No.: PCT/IL2017/050795

§ 371 Date: Jan. 10, 2019

Related U.S. Application Data
(60) Provisional application No. 62/361,046, filed on Jul. 12, 2016.

(51) Int. Cl. C12M 1/09; C12M 1/00; C12M 1/04; C12N 1/12; A23L 33/10; A23K 10/18

(52) U.S. Cl. CPC C12M 23/56; C12M 23/34; C12M 23/22; C12M 23/02; A23K 10/18; C12M 23/24; C12N 1/12; A23L 33/10; C12M 21/02

(57) ABSTRACT

A floatable composition comprising obligate photoautotrophic microalgae and a floating element is provided. Also provided is a compartmentalized composition comprising at least two compartments wherein a first compartment of the at least two compartments comprises an obligate photoautotrophic microalgae and a second compartment of the at least two compartments comprises an obligate heterotrophic or mixotrophic microalgae, the compartments are designed of a structure and/or composition ensuring symbiosis between the obligate photoautotrophic microalgae and the obligate heterotrophic or mixotrophic microalgae.

### FIELD AND BACKGROUND OF THE INVENTION

The present invention, in some embodiments thereof, relates to compositions comprising microalgae and methods of producing and using same.

Algae refer to a large, diverse group of photosynthetic organisms including unicellular genera, such as Chlorella and the diatoms, to multicellular forms. Most are aquatic and autotrophic and lack many of the distinct cell and tissue types, such as stomata, xylem and phloem, which are found in land plants.

Algae have been used as food, feed and fertilizer for centuries. In the 1950's algae were considered a candidate for protein supply for the increasing world population. Algae grow quickly and abundantly in all kinds of water, and contain high levels of various compounds that can be used for renewable fuel, animal feed, cosmetics, fertilizer, drug delivery, nutraceuticals, water purification, bioplastic, lubricants and human and animal food including health beneficial compounds such as antioxidants, omega-3 oil, carbohydrates, sugars proteins, etc.

Microalgae constitute a source of active compounds such as β-carotene from Dunaliella salina, Astaxanthin, cantaxanthin, lutein from Haematococcus pluvialis, Cantaxanthin, astaxanthin from Chlorella vulgaris, Canthaxanthin, astaxanthin, β-carotene from Coelastrella striolata var. multistriata, Lutein, β-carotene from Scenedesmus almeriensis, DHA from Cryptheconidium, EPA Odontella, Vitamin B12 from Spirulina, etc.

Microalgae or unicellular algae perform a wide range of functions, such as algae growth, decomposition of organic matter, anti bacterial water protection, detoxification of heavy metals and anti oxidation as part of environmental remediation.

For example, microalgae such as Chlorella, Dunaliella and Spirulina that are known to be rich in more than 20 different vitamins, amino acids and minerals, are abundant in beta-carotene and chlorophyll, as well as growth factors. Chlorella, is rich in high quality proteins (50-60% of total mass), carbohydrate (15-20%), fat (10-15%), minerals (6%) and 4% moisture. In addition it also contains Vitamin B12 and Growth factor shown to stimulate tissue repair and promote the growth of children and animals. Chlorella has also been reported to stimulate the immune system, displays antioxidant and anti tumor activity, exhibits anti-aging properties, and more. Dunaliella algae contain proteins, lipids, sugars and minerals as well as vitamins and a variety of physiologically active ingredients, especially β-carotene. Dried powder of microalgae, such as Dunaliella is granulated together with other materials and encapsulated in a hard capsule that is commercially available.

However, preparation and preservation of various microalgae including Chlorella, Dunaliella or Spirulina either in tablets, granules or in liquid extract may result in destruction of most of the physiologically active ingredients and thus lower their beneficial effect.

In order to preserve the maximal and optimal level of beneficiary microalgae products, viable microalgae should be available in their natural form such as by production of encapsulated viable microalgae. Such products can serve as vegetarian food stuff containing entrapped viable algae of one or more species either by concomitant culturing or by compartmentalization of the different species in various edible polymers of different structures.

Encapsulated microalgae have been described for several purposes such as feed, cosmetics, as well as oxygen producers for co-cultured heterotrophic cells (Bloch K, Papismedov E, Yavriyants K, Vorobeychik M, Beer S, Vardi P. Photosynthetic oxygen generator for bioartificial pancreas. Tissue Eng. 2006 February; 12(2):337-44. Kitcha S, Cheirsilp B. Enhanced lipid production by co-cultivation and co-encapsulation of oleaginous yeast Trichosporonoides spathulata with microalgae in alginate gel beads. Appl Biochem Biotechnol. 2014 May; 173(2):522-34. doi:10.1007/s12010-014-0859-5. de-Bashan L E, Bashan Y. Joint immobilization of plant growth-promoting bacteria and green microalgae in alginate beads as an experimental model for studying plant-bacterium interactions. Appl Environ Microbiol. 2008 November; 74(21):6797-802. doi: 10.1128/AEM.00518-08).

Encapsulated microalgae have been shown to preserve microalgae growth rate and even accelerate cell proliferation and microalgae compounds production (Joo D S, Cho M G, Lee J S, Park J H, Kwak J K, Han Y H, Bucholz R. New strategy for the cultivation of microalgae using microencapsulation. J Microencapsul. 2001 September-October; 18(5):567-76. de-Bashan L E, Bashan Y. Immobilized microalgae for removing pollutants: review of practical aspects. Bioresour Technol. 2010 March; 101(6):1611-27. doi: 10.1016/j.biortech.2009.09.043). Such a possibility is of crucial advantage in the food industry as well as in any other algae based industrial systems.

Additional background art includes:

U.S. Pat. No. 9,090,885

U.S. Pat. No. 8,012,500.

### SUMMARY OF THE INVENTION

According to an aspect of some embodiments of the present invention there is provided a floatable composition comprising obligate photoautotrophic microalgae and a floating element.

According to an aspect of some embodiments of the present invention there is provided a compartmentalized composition comprising at least two compartments wherein a first compartment of the at least two compartments comprises an obligate photoautotrophic microalgae and a second compartment of the at least two compartments comprises an obligate heterotrophic or mixotrophic microalgae, the compartments are designed of a structure and/or composition ensuring symbiosis between the obligate photoautotrophic microalgae and the obligate heterotrophic or mixotrophic microalgae.

According to some embodiments of the invention, the first compartment of the at least two compartments is transparent to light and wherein when the second compartment comprises mixotrophic microalgae the second compartment is non-transparent to light.

According to some embodiments of the invention, the composition as described herein allows free diffusion of small molecules, minerals and gas between the at least two compartments.

According to some embodiments of the invention, the composition as described herein is formulated as a capsule.

According to some embodiments of the invention, the capsule is shaped as a fiber or a sphere.

According to some embodiments of the invention, a concentration of the obligate photoautotrophic microalgae in the capsule is 10⁶-10¹⁰ cells/cm³ capsule.

According to some embodiments of the invention, a concentration of the obligate heterotrophic or mixotrophic microalgae in the capsule is 10⁶-10¹⁰ cells/cm³ capsule.

According to some embodiments of the invention, the microalgae are viable.

According to some embodiments of the invention, the capsule is 0.1-20 mm in diameter.

According to some embodiments of the invention, the obligate photoautotrophic microalgae are at present in the composition in at least 90% purity.

According to some embodiments of the invention, the obligate photoautotrophic microalgae are at present in the first compartment in at least 90% purity and the obligate heterotrophic or mixotrophic microalgae are present in the second compartment in at least 90% purity.

According to some embodiments of the invention, the composition further comprises a floating element rendering the composition floatable.

According to some embodiments of the invention, the composition as described herein is ingestible by an organism.

According to some embodiments of the invention, the organism is a human being.

According to some embodiments of the invention, the organism is a non-human animal.

According to some embodiments of the invention, the composition as described herein is edible.

According to some embodiments of the invention, the first compartment encapsulates the second compartment.

According to some embodiments of the invention, the obligate photoautotrophic microalgae are selected from the group consisting of Dunaliella sp., Nannochloropsis sp., Synechococcus sp. and Spirulina sp.

According to some embodiments of the invention, the heterotrophic microalgae or the mixotrophic microalgae are characterized by growth rate faster than that of the obligate photoautotrophic microalgae.

According to some embodiments of the invention, the obligate heterotrophic microalgae are selected from the group consisting of Schizochytrium sp. and Crypthecodinium sp.

According to some embodiments of the invention, the mixotrophic microalgae are selected from the group consisting of Chlorella sp. and Chlamydomonas sp.

According to some embodiments of the invention, the mixotrophic microalgae are from the group of Chlorella sp. and the obligate photoautotrophic microalgae are from the group of Spirulina sp.

According to some embodiments of the invention, the microalgae are genetically modified.

According to some embodiments of the invention, the second compartment comprises an additive which affects turbidity.

According to some embodiments of the invention, the additive is selected from the group consisting of a pigment, a colorant, a dye and a protein.

According to some embodiments of the invention, the obligate photoautotrophic microalgae are encapsulated by a polymeric material.

According to some embodiments of the invention, the first compartment and the second compartment are composed of polymeric materials.

According to some embodiments of the invention, the polymeric material is light transparent.

According to some embodiments of the invention, the polymeric material is selected from the group consisting of alginate, agarose, gelatin and chitosan.

According to an aspect of some embodiments of the present invention there is provided a method of producing a nutritional composition, the method comprising:

(a) formulating obligate autotrophic microalgae and optionally obligate heterotrophic microalgae into a composition comprising a floatable element, wherein the formulating is effected under conditions that maintain viability of the microalgae; and

(b) culturing the microalgae in the composition, thereby producing the nutritional composition.

According to an aspect of some embodiments of the present invention there is provided a method of producing a nutritional composition, the method comprising:

(a) producing a compartmentalized composition comprising at least two compartments wherein a first compartment of the at least two compartments comprises an obligate photoautotrophic microalgae and a second compartment of the at least two compartments comprises an obligate heterotrophic or mixotrophic microalgae, the compartments are designed of a structure and/or composition ensuring symbiosis between the obligate photoautotrophic microalgae and the obligate heterotrophic or mixotrophic microalgae; and

(b) culturing the microalgae in the particles, thereby producing the nutritional composition.

According to some embodiments of the invention, the first compartment of the at least two compartments is transparent to light and wherein when the second compartment comprises mixotrophic microalgae the second compartment is non-transparent to light.

According to some embodiments of the invention, the compartmentalized composition allows free diffusion of small molecules, minerals and gas between the at least two compartments.

According to some embodiments of the invention, the composition is formulated as a capsule.

According to some embodiments of the invention, the capsule is shaped as a fiber or a sphere.

According to some embodiments of the invention, a concentration of the obligate photoautotrophic microalgae in the capsule is 10⁶-10¹⁰ cells/cm³ capsule.

According to some embodiments of the invention, the microalgae are viable in the composition.

According to some embodiments of the invention, the capsule is 0.1-20 mm in diameter.

According to some embodiments of the invention, the obligate photoautotrophic microalgae are at present in the composition in at least 90% purity.

According to some embodiments of the invention, the obligate photoautotrophic microalgae are at present in the first compartment in at least 90% purity and the obligate heterotrophic or mixotrophic microalgae are present in the second compartment in at least 90% purity.

According to some embodiments of the invention, the composition further comprises a floating element rendering the composition floatable.

According to some embodiments of the invention, the composition is ingestible by an organism.

According to some embodiments of the invention, the composition is edible.

According to some embodiments of the invention, the first compartment encapsulates the second compartment.

According to some embodiments of the invention, the first compartment is composed of a first polymer and the second compartment is composed of a second polymer and the producing is effected by dropping or electrospinning a first polymeric solution comprising the first polymer and the obligate photoautotrophic microalgae and a second polymeric solution comprising the second polymer and the obligate heterotrophic or mixotrophic microalgae into a polymerizing solution.

According to some embodiments of the invention, the dropping or electrospinning the first polymeric solution and the second polymeric solution is from co-axial nozzles or non-co-axial nozzles.

According to some embodiments of the invention, the method further comprises isolating the microalgae following the culturing.

According to some embodiments of the invention, the culturing comprises outdoor.

According to some embodiments of the invention, the culturing comprises indoor.

According to some embodiments of the invention, the culturing comprises open settings.

According to some embodiments of the invention, the culturing comprises closed settings.

According to some embodiments of the invention, the method further comprises storing the microalgae for 1 month e.g., 3 months to 12 months under conditions that maintain viability of the microalgae.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of a compartmentalized composition according to some embodiments of the invention.

FIG. 2 shows pictures of capsules composed of transparent peripheral compartment for obligatory photoautotrophic microalgae (Spirulina) and central nontransparent compartment/s for mixotrophic/heterotrophic microalgae (Chlorella).

FIGS. 3A-B are images showing alginate beads composed of Spirulina and Chlorella cells (FIG. 3A) and alginate beads composed of spirulina cells alone (FIG. 3B) after about one month in darkness at room temperature.

### DESCRIPTION OF SPECIFIC EMBODIMENTS OF THE INVENTION

The present invention, in some embodiments thereof, relates to compositions comprising microalgae and methods of producing and using same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Microalgae are an important source of proteins, minerals, vitamins and antioxidants in human and animal nutrition. Among many challenges faced in the commercial cultivation of microalgae, low-cost water and nutrients availability is crucial.

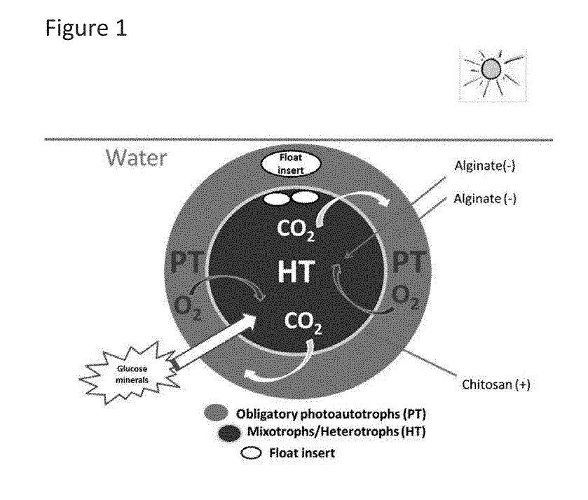

Whilst reducing the present invention to practice, the present inventors have devised a novel approach for the co-culturing of microalgae, resulting in a complex composition of high nutritional value that can be obtained at low cost without risking losing one of the microalgal species. The approach is based on compartmentalization of encapsulated microalgae providing enhanced production of high quality living algal biomass due to mutual symbiosis between co-cultivated algae that allow efficient transfer of carbon dioxide and oxygen between co-cultivated microalgal species; prevention of selective elimination of one of co-cultivated algal species, and production of diversified desirable microalgae-derived products in the same product (e.g. capsule, see FIG. 1).

Thus, according to an aspect of the invention there is provided a compartmentalized composition comprising at least two compartments, wherein a first compartment of the at least two compartments comprises an obligate photoautotrophic microalgae and a second compartment of the at least two compartments comprises an obligate heterotrophic or mixotrophic microalgae, the at least two compartments being designed of a structure and/or composition ensuring symbiosis between the obligate photoautotrophic microalgae and the obligate heterotrophic or mixotrophic microalgae.

According to another aspect of the invention there is provided a floatable composition comprising obligate photoautotrophic microalgae and a floating element.

The composition can be compartmentalized or non-compartmentalized.

As used herein “microalgae” refers to microscopic algae, typically found in freshwater and marine systems living in both the water column and sediment. Microalgae are unicellular species which exist individually, or in chains or groups.

As used herein “obligate photoautotrophic microalgae” or “obligate phototroph microalgae” is a microalgal species that requires light energy for the production of chemical energy and is incapable of using exogenously supplied performed organic molecules as its sole source of carbon or energy.

As used herein a “heterotroph” is a microalgal species that can use preformed organic compounds as the source of carbon and energy in the absence of light. Heterotrophs can, therefore, grow independently of illumination; for example, heterotrophs can grow in the dark, in the light, and in partial light. Similarly, “heterotrophic growth” refers to growth which does not require light to occur and can, therefore, occur independent of the level or lack of illumination.

The heterotroph can be obligate heterotrophic microalgae or a mixotrophic microalgae.

As used herein “obligate heterotrophic microalgae” refers to a microalgal species that uses preformed organic compounds and not light as the source of carbon and energy. Obligate heterotrophs, therefore, grow independently of illumination; for example, heterotrophs can grow in the dark, in the light, and in partial light. Similarly, “heterotrophic growth” refers to growth which does not require light to occur and can, therefore, occur independent of the level or lack of illumination.

As used herein “a mixotrophic microalgae” refers to a microalgal species that that can use a mix of different sources of energy and carbon, e.g., photo- and chemotrophy.

According to a specific embodiment, the heterotrophic microalgae or the mixotrophic microalgae are characterized by growth rate faster than that of the obligate photoautotrophic microalgae under the optimal growth conditions for each species (Yu-Ru Li, Wen-Tien Tsai, Yi-Chyun Hsu, Meng-Zhi Xie, Jen-Jeng Chen. Comparison of autotrophic and mixotrophic cultivation of green microalgal for biodiesel production. Energy Procedia, 2014, 52, 371-376; Perez-Garcia O, Escalante F. M. E, de-Bashan L. E, Bashan Y. Heterotrophic cultures of microalgae: Metabolism and potential products. Water research, 2011, 45, 11-36).

Advancements in algal molecular biology have made it possible to genetically modify autotrophs to heterotrophs. Methods of performing these modifications are described in U.S. Pat. No. 7,939,710, which is hereby incorporated by reference in its entirety.

Generally, the present teachings relate to naïve or genetically modified microalgae.

According to an embodiment, at least one of the obligate autotrophic microalgae, obligate heterotrophic microalgae or the mixotrophic microalgae is genetically modified.

The genetic modification can be done to improve the cultivation of the microalgae (e.g., survival, tolerance to abiotic/biotic stress, biomass).

Alternatively or additionally, the genetic modification can be done to improve the quality of the product (e.g., nutritional value, therapeutic value, energetic value, digestibility).

Lists of obligate photoautotrophic microalgae may be found in a review by Droop (Droop M R “Heterotrophy of Carbon.” In Algal Physiology and Biochemistry, Botanical Monographs, 10: 530-559, ed. Stewart W D P, University of California Press, Berkeley (1974)); and a representative, non-exclusive list of obligate photoautotrophic microalgae with potential or known commercial value is provided below (Table 1, grouped at the phylum level. The “common name” is in parenthesis.

TABLE 1

| | |
|---|---|
| Cyanophyta (Blue-green | Spirulina, Anabaena. |
| algae) | |
| Chlorophyta (Green algae) | Dunaliella, Chlamydomonas, |
| | Heamatococcus. |
| Rhodophyta (Red algae) | Porphyridium, Porphyra, Euchema, |
| | Graciliaria. |
| Phaeophyta (Brown algae) | Macrocystis, Laminaria, Undaria, Fucus. |
| Baccilariophyta (Diatoms) | Nitzschia, Navicula, Thalassiosira, |
| | Phaeodactylum. |

According to a specific embodiment, the obligate phototrophic microalgae is selected from the group consisting of Dunaliella sp., Nannochloropsis sp., Synechococcus sp. and Spirulina sp.

Non limiting examples of obligate heterotrophic microalgae are selected from the group consisting of Schizochytrium sp. and Crypthecodinium sp.

Non limiting examples of mixotrophic microalgae are selected from the group consisting of Chlorella sp. and Chlamydomonas sp.

It will be appreciated that the composition may comprise a plurality of species from each of obligate heterotrophic microalgae, mixotrophic microalgae and obligate phototrophic microalgae, dependent upon compliance to co-culturing requirements (in the case of the compartmentalized composition).

According to a specific embodiment the obligate phototrophic microalgae is Spirulina sp. and the mixotrophic microalgae is Chlorella sp.

As used herein “symbiosis” in this case refers to mutualistic symbiosis in which case the obligate autotroph provides oxygen to the obligate heterotrophic or mixotrophic microalgae; while the obligate heterotrophic or mixotrophic microalgae provide the obligate autotroph with carbon dioxide.

As used herein “compartmentalized composition” refers to a composition comprising at least two or more compartments.

A compartment refers to separate division or section and can take a variety of forms, geometries, and shapes, e.g. it can be a well, chamber, channel, droplet, bead, plug, etc.

According to a specific embodiment, the first compartment of the at least two compartments is transparent to light and wherein when the second compartment comprises mixotrophic microalgae. The second compartment is non-transparent to light, to reduce the dependency of the latter on photosynthesis.

Alternatively or additionally, the first compartment encapsulates the second compartment, such that said second compartment constitutes a core while the first compartment constitutes a coat or a shell that prevents light penetration in to the second compartment.

Alternatively, or additionally the first compartment of the at least two compartments is transparent to light and wherein when the second compartment comprises mixotrophic microalgae. The second compartment is non-transparent to light and the compartments are oriented juxtaposing each other to allow passage of minerals and or gases (allowing symbiosis), yet the second compartment can still be exposed to light.

However, in order to ensure maximal photosynthesis by the olbligate photoautotroph and minimal photosynthesis by the heterotroph, the composition is designed of both structure (core and shell) and composition (e.g., transparent first compartment and non-transparent second compartment and/or said second compartment comprising an additive that affects the turbidity) that serve this purpose.

According to a specific embodiment the composition (compartmentalized and/or floatable) is formulated as a capsule, granule, particle, bubbles or droplets.

According to a specific embodiment, the composition is shaped as a sphere.

According to a specific embodiment, the composition is shaped oval shaped.

According to a specific embodiment, the composition is shaped as a fiber.

According to a specific embodiment, each compartment provides for a confined area where a particular type of microalgae may be cultured without being intermixed with a different type of microorganism (e.g., bacteria, fungi or another type of microalgae which presence is not desired).

The compartmentalized composition thus provides for flow of gases, glucose and minerals e.g. oxygen from the obligate autotrophs to the obligate heterotrophs or mixotrophs, or carbon dioxide from the obligate heterotrophs or mixotrophs to the obligate autotrophs between the different compartments. However, the compartmentalized composition is designed such that it does not allow passage of cells between the compartments. An exemplary embodiment is depicted in FIG. 1.

The composition of some embodiments of the invention is designed such that it allows the substantiation of symbiosis between different types of algae residing in the different compartments.

According to a specific embodiment, the compartmentalized composition is devoid of barriers and/or channels (discrete of the materials composing each compartment).

In other embodiments, the compartments are separated from one another by the presence of a barrier which still allows the free passage of small molecules (e.g. glucose, CO₂, O₂, minerals) but not cells between the compartments.

Thus, a plurality of (two or more) compartments provide culturing space of two or more different species of microalgae (e.g., obligate autotroph and obligate heterotroph) without any cross-contaminations with each other or by a “third” party (contaminant, e.g., bacteria). Localized growth of a predetermined algal type (e.g. strain, species) in a single compartment is important for the confined culturing method of the compositions of some embodiments of the invention.

According to an embodiment of the invention, the composition comprises a floatable composition comprising obligate photoautotrophic microalgae and a floating element.

Such a composition may be compartmentalized.

According to an alternative embodiment, the floatable composition is non-compartmentalized.

As used herein a “floating element” refers to an element that imparts the composition comprising the microalgae (compartmentalized or non-compartmentalized) with buoyancy in the microalgae culture medium. The floating element is designed such that the particle is still submerged to allow algae growth (e.g., a-concentric floating element). The floating element forms a part of the composition, whereby it can be mixed with the polymer used for cell immobilization. The selection of the floating element much depends on the type of medium used for microalgal culturing (e.g., fresh water, waste water, brine, sea water etc.) as the salt concentration affects the buoyancy of the composition. For example, the floating elements may be particles made of natural or artificial edible light polymers such as natural or artificial edible polymers, wax, air bubbles, oil droplets, aromatic oil, etc. Current medical applications of floating capsules made from biopolymers can be found in these reviews: Lopes C M, Bettencourt C, Rossi A, Buttini F, Barata P. Overview on gastroretentive drug delivery systems for improving drug bioavailability. Int J Pharm. 2016 May 9. pii: S0378-5173(16)30386-6. doi: 10.1016/j.ijpharm.2016.05.016. [Epub ahead of print] Review. Kau shik AY, Tiwari A K, Gaur A. Role of excipients and polymeric advancements in preparation of floating drug delivery systems. Int J Pharm Investig. 2015 January-March; 5(1):1-12. doi: 10.4103/2230-973X.147219. Review, each of which is incorporated by reference in its entirety.

The composition can be designed of any chemical composition, size or structure.

However, according to a specific embodiment, the composition is designed such that it is ingestible by a human or non-human animal.

Thus, dependent on the intended use, the composition is designed of a chemical (e.g., polymer(s)) composition and/or dimensions suitable for being ingestible.

As used herein “ingestible” refers to taken as a food by an organism, e.g., human being, although veterinary applications are also contemplated.

Thus according to a specific embodiment, the composition is of an ingestible size or texture.

According to a specific embodiment, the capsule is 0.1-20 mm in diameter. For example, for human consumption the composition (e.g., capsule or other formulation described herein) is 1-5 mm in diameter.

According to a specific embodiment, the composition is edible (fits to be eaten e.g., by human beings). According to a specific embodiment, the composition is composed of materials that are approved by regulatory agencies for being consumed by the end subject (e.g., human being), such as by the FDA.

According to a specific embodiment, the first compartment of the at least two compartments is transparent to light and wherein when the second compartment comprises mixotrophic microalgae the second compartment is non-transparent to light.

As used herein “transparent to light” allows the passage of visible light without being scattered. Thus, a compartment being transparent to light refers to a transparency level, which ascertains that light is not a limiting factor for photosynthesis.

As used herein “non-transparent to light” ensures that light is a limiting factor for mixotrophic microalgae in said second compartment.

Various materials which are transparent to the visible light (400-700 nm) are known. These include but are not limited to gelatin, alginate, chitosan and/or agarose.

Various materials which are non-transparent to the visible light are known. These include, but are not limited to natural or synthetic pigments, food colorants and activated charcoal.

It will be appreciated that the second compartment may be rendered less transparent to the visible light by adding an additive that affects the turbidity of a culture medium present in the second compartment.

These include, but are not limited to, a pigment, a dye, a colorant and a protein. Many such light absorbent additives are known in the art (Aberoumand A. A Review Article on Edible Pigments Properties and Sources as Natural Biocolorants in Foodstuff and Food Industry. World Journal of Dairy & Food Sciences, 2011, 6 (1): 71-78, which is hereby incorporated by reference in its entirety). Each of such additives is selected such that it does not interfere with the growth of the microalgae in the compartments.

In order to ensure growth of the microalgae in the composition, the composition (floatable and/or compartmentalized) comprises a culture medium or culturing constituents. All materials used for compartmentalization are permeable for small molecules (glucose, minerals, gases) required for growth of microalgae.

Methods of algal culturing are well known in the art.

As used herein “culture medium” refers to a solid, liquid or gel medium (gel and solid may become liquid upon culturing) that provides the microalgae with sufficient nutritional support to mediate survival (maintains viability without expansion) or even growth (expansion/proliferation). According to a specific embodiment the culture media are designed to promote growth of one microalgal species and survival (e.g., starvation medium) of another present in the composition. Alternatively, the media may be selected promoting growth of all microalgal species in the composition. Alternatively, the media may be selected promoting survival of two species.

The choice of medium used will depend on several factors: the growth requirements of the microalgae, how the constituents of the medium affect the final product quality, and the cost. Since according to some embodiments of the invention, the product is for the food industry, food grade chemicals are used. For animal market non-feed grade materials may be used, however care should be taken not to include various contaminants such as heavy metals.

The composition of the culture medium may differ when different types of microalgae are cultured. The culture medium may optionally be modified (e.g. some compounds may be omitted from the culture medium when one wants to starve the microalgae, or one wants to apply selection pressure).

According to some embodiments of the invention, the first compartment and the second compartment comprise an identical culture medium.

Compositions of the present invention comprise viable microalgae.

Thus according to a specific embodiment, at least 90% of the algae of each population present in the composition (compartmentalized or non-compartmentalized) is viable following 3 months in culture.

According to a specific embodiment, the viability is maintained about the same even after culturing and storage for at least 6 months at 4-8° C.

Methods of determining microalgae viability comprise staining with methyl-thiazolyl-tetrazolium (MTT), Evans Blue, and Neutral Red (Da Luz et al. Efficiency of Neutral Red, Evans Blue and MTT to assess viability of the freshwater microalgae Desmodesmus communis and Pediastrum boryanum. Phycological Research, 2016; 64: 56-60 doi: 10.1111/pre.12114, which is hereby incorporated by reference in its entirety).

Viability of the microalgae in the end product ensures the provision of an edible product with super beneficial health value with a wide range of natural fresh compounds. The final product contains entrapped viable algae e.g., of both autotrophic and heterotrophic families either of pure or mixed microalgae communities. Such end product of microalgae offers a significant increase in the number of health beneficial products derived from various pure or mixed microalgae source of both autotrophic and heterotrophic origin.

The purity of each population in the composition may vary. However, according to a specific embodiment, the obligate photoautotrophic microalgae are present in the composition in at least 90%, 95%, 97% or even 100% purity.

According to another embodiment, the obligate photoautotrophic microalgae are present in the first compartment in at least 90%, 95%, 97% or even 100% purity and the obligate heterotrophic or mixotrophic microalgae are present in the second compartment in at least 90%, 95%, 97% or even 100% purity.

It will be appreciated that the obligate photoautotrophic microalgae may comprise a single species or strain of obligate photoautotrophic microalgae or a plurality (i.e., two or more) species or strains of obligate photoautotrophic microalgae.

The same holds for the obligate heterotrophic or mixotrophic microalgae.

Thus, according to an aspect of the invention there is provided a method of producing a nutritional composition, the method comprising:

(a) formulating obligate autotrophic microalgae and optionally obligate heterotrophic microalgae into a composition comprising a floatable element, wherein the formulating is effected under conditions that maintain viability of the microalgae; and

(b) culturing the microalgae in the particles, thereby producing the nutritional composition.

According to an alternative aspect there is provided a method of producing a nutritional composition, the method comprising:

(a) producing a compartmentalized composition comprising at least two compartments wherein a first compartment of the at least two compartments comprises an obligate photoautotrophic microalgae and a second compartment of the at least two compartments comprises an obligate heterotrophic or mixotrophic microalgae, the compartments are designed of a structure and/or composition ensuring symbiosis between the obligate photoautotrophic microalgae and the obligate heterotrophic or mixotrophic microalgae; and

(b) culturing the microalgae in the particles, thereby producing the nutritional composition.

In addition to nutritional value, many microalgae strains (e.g. Chlorella, Dunaliella, Spirulina express detoxification properties allowing use of edible microalgae for removal of heavy metals from human body (Kaplan, D. Absorption and Adsorption of Heavy Metals by Microalgae, in Handbook of Microalgal Culture: Applied Phycology and Biotechnology, 2013, Second Edition (eds A. Richmond and Q. Hu), John Wiley & Sons, Ltd, Oxford, UK. doi: 10.1002/9781118567166.ch32; Bobrov Z., Tracton I., Taunton K., Mathews M. Effectiveness of whole dried Dunaliella salina marine microalgae in the chelating and detoxification of toxic minerals and heavy metals. Hence the nutritional compositions described herein also have therapeutic or prophylactic properties.

It will be appreciated that any embodiment or combination of embodiments described herein with respect to the composition, also applies to the method of manufacturing.

According to an embodiment, the method further comprising isolating the microalgae following the culturing.

According to a specific embodiment, isolating is by filtration, centrifugation, magnetic field, chemical coagulation/flocculation, auto and bioflocculation, gravity sedimentation (Barros et al. Harvesting techniques applied to microalgae: A review. Renewable and Sustainable Energy Reviews. 2015, 41, 1489-1500, which is hereby incorporated by reference in its entirety).

Methods of compartmentalization are provided infra. Such methods can also be used for preparing a non-compartmentalized composition e.g., a non-compartmentalized capsule. A specific embodiment for preparing a non-compartmentalized composition (Step 1) and compartmentalized compositions (Step 2) is provided in the Examples section which follows. By no means is this description is aimed to be limiting in terms of reagents, algal species used and is considered a part of the instant specification.

Thus for instance, the compartmentalized composition can be made by dropping.

Accordingly, the first compartment is composed of a first polymer and the second compartment is composed of a second polymer and the producing is effected by dropping a first polymeric solution comprising the first polymer and the obligate photoautotrophic microalgae and a second polymeric solution comprising the second polymer and the obligate heterotrophic or mixotrophic microalgae into a polymerizing solution.

According to a specific embodiment, dropping said first polymeric solution and said second polymeric solution is from co-axial nozzles or non-co-axial nozzles.

Another method of forming (e.g., barrier-free, channel-free) a compartmentalized composition that can be used according to the present teachings comprises electrospinning.

An exemplary embodiment of the method is described in WO2009/104174 which is hereby incorporated by reference describes compartmentalized tubular structures comprising a core and a shell, that may comprise viable cells.

According to another embodiment, the compositions may be formulated as droplets, gel microdroplet (GMD), beads or plugs.

In general, a “droplet” refers to a relatively small volume of material. Droplets according to this invention can be polymeric or solid particles, gel microdroplets, beads, or plugs.

Suitable droplets may have different shapes and sizes. The droplets may have different sizes and geometries, and may be symmetric or asymmetric. For example, droplets may refer to a single sphere or oval, may refer to a core-shell configuration, to a group of smaller particles attached together (e.g. to form grape-like structure), to a string of particles some of which are in contact with each other, etc.

Droplets may contain two or more, if desired multiple, types of microalgae or colonies of microalgae. Each type of microalgae may be positioned anywhere in or on the droplets (so long as at least one type is confined from at least one other type). Alternatively, microalgae may be encapsulated in the droplets.

Gel Microdroplets

A “gel microdroplet” (GMD) (also referred to herein as a gel bead, or a gel particle) refers to very small droplets, i.e. very small volume entities comprised of gel (and optionally liquid) material, and which can contain zero, one or multiple biological entities. For example, two or more types of microalgal species may be encapsulated in agarose GMDs. In particular, aqueous droplets containing microalgae, growth media, and liquid agarose may be formed in fluorinated oil. The GMDs may optionally contain inorganic and/or organic chemical compounds; these compounds may optionally be in solution. GMDs have volumes which may be defined by a boundary comprised of another liquid, such as a non-aqueous fluid, or by a permeability barrier such as a membrane, such that the membrane is capable of retaining biological entities (e.g. microalgae) of interest within a GMD, and also capable of passing other biological entities such as molecules (smaller than microalgae). For example, it would be possible to generate two or more streams of two or more different microalgal strains, then combine them into a single droplet, and then polymerize it into a GMD (e.g. agarose GMD), where the microalgal strains are compartmentalized and spatially separated. Although GMDs can be of any shape, GMDs are often approximately spherical because of the tendency of forces associated with the boundaries of GMDs to round up the deformable GMDs. Other forces, for example hydro-dynamic shear associated with stirring a GMD suspension, adhesion to a surface, or gravity, tend to cause departure from a spherical shape. Further, GMDs which contain or occupied by entities whose volume is a relatively large fraction of the GMD volume can result in GMDs which are non-spherical. Thus, for example, cell or a population of cells surrounded by a thin gel coating (and optionally with an aqueous solution), which in turn is surrounded by a non-aqueous fluid, is a GMD. Similarly, a non-biological particle is surrounded by a thin gel coating (and optionally with an aqueous solution), which in turn is surrounded by a non-aqueous fluid, is also a GMD.

Various types of gels can be used in the practice of the invention. They include: standard gel, when growth and potential of mixing of microalgae is slow or is not a concern; gels that are impermeable to microalgae, so the microalgae do not move through the gel; and arbitrary gels, where the interfaces among the gels have membranes impermeable to microalgae, yet permeable to desired chemicals. When generating gel beads, such membranes could be formed chemically as the beads are being made, e.g. by reacting two polymers on the surface of the bead, or by incorporating those two polymers into the individual gels, so at interfaces of gels membranes form. The formation of gel or polymeric substances in a plugs could also be initiated by an externally by light, temperature change, additional of a small molecule, pH change, pressure change, contact with carrier fluid, or contact with channel walls.

Beads

In another embodiment, the droplets contains two or more beads. Each bead can be of the same or different type, shape, and size. Beads may be connected. The beads may be gel beads, for example they may be agarose beads. For example, layered beads may be generated, where each layer will have discrete types of microalgae. In some examples, by making beads magnetic, the beads can be distributed in the desired area, e.g. environment, and then easily picked up when desired, for example, by using an electromagnet or a permanent magnet, when the beads are no longer needed.

Plugs

Droplets can be liquid (usually aqueous) which exists either in a two-phase system (e.g., organic phase/aqueous phase, fluorous phase/aqueous phase) or in a single phase with an emulsifying agent/surfactant (e.g., aqueous droplets surrounded by aqueous bulk solution). A “plug” is a specific type of droplet (Song et al., 2006, Angew. Chem. Int. Ed. 45: 7336-7356; Chen et al., 2006, Curr. Opin. Chem. Bio. 10: 226-231).

Formation of liquid plugs was previously described by this inventor in U.S. Pat. No. 7,129,091. In the present invention, different types of microalgae are introduced into different plug fluids.

Methods of incorporating multiple and different microalgae into a spatially structured plug include combination of fluids containing microalgae with fluids containing components necessary to form a gel or a polymer or a solid matrix. Upon forming the plug, the different types of microalgae would have a non-uniform spatial distribution throughout the plug and this initial spatial distribution can be controlled using microfluidic techniques such as laminar flow of multiple streams. Before the microalgae are able to substantially intermix, the components would form a gel or a polymer or a solid matrix and prevent significant further intermixing of the microalgae. In this way, the non-uniform distribution of the microalgae in the plug would be preserved. Formation of a gel or a polymer or a solid matrix could be accomplished in a number of ways, including spontaneous formation, as takes place when a supercooled gel or solid transitions from a liquid state into a gel or solid state; stimulation formation, as takes place when pressure, temperature or UV or visible light or another form of radiation is applied, or a chemical reagent is added. Chemical reagents include cross-linking agents, changes in pH, change in ionic composition, or the additional of a small molecule, ions, or a macromolecule. Chemical reagents may be pre-loaded into the plug fluids, or added after the formation of a plug.

In addition, methods of incorporating multiple and different microalgae into a spatially structured plug include sequentially forming layers containing microalgae.

Regardless of the formulation, the microalgae are cultured using methods which are well known in the art.

Culture temperatures may vary from about 4° C. to temperatures reaching even about 40° C., according to specific microalgae requisite. The culturing period may depend on the type of microalgae and its end use. Termination of culturing depends on the algae strain and is within the skills of the skilled artisan. Culturing can be effected in open settings (e.g., open ponds) or closed settings (e.g., fermentors) using natural or artificial light for photosynthesis. Following is a brief description of such culturing settings.

Most commercial production techniques use large open ponds, taking advantage of natural sunlight, which is free. These systems have a relatively low surface area to volume ratio with corresponding low cell densities. Hence in such systems impellers are typically used to benefit the entire photosynthetic depth or the water column. The need to exclude contaminating organisms in open ponds, typically restricts the usefulness of open ponds to a limited number of algae that thrive in conditions not suitable for the growth of most organisms. For example, Dunaliella salina can be grown at very high salinities. Apt K E et al, “Commercial Developments in Microalgal Biotechnology,” J Phycol. 35:215-226 (1999). Of course measures are taken to comply the salinity conditions for other species used in the composition.

Enclosed photobioreactors, such as tubular photobioreactors, are an alternative outdoor closed culture technology that utilize transparent tubes enclosing the culture minimizing contamination. They provide a very high surface to volume ratio, so cell densities are often much higher than those that can be achieved in a pond.

Numerous designs have also been constructed for the indoor, closed culture of algae using electric lights for illumination. Ratchford and Fallowfield (1992) “Performance of a flat plate, air lift reactor for the growth of high biomass algal cultures,” J Appl. Phycol. 4: 1-9; Wohlgeschaffen, G D et al. (1992) “Vat incubator with immersion core illumination a new, inexpensive set up for mass phytoplankton culture,” J Appl. Phycol. 4:25-9; Iqbal, M et al. (1993) “A flat sided photobioreactor for culturing microalgae,” Aquacult. Eng. 12:183-90; Lee and Palsson (1994) “High-density algal photobioreactors; using light-emitting diodes,” Biotechnol. Bioeng. 44:1161-7.

Once culturing is terminated (e.g., actively) the algae are isolated from the culture.

This can be done by filtration, use of magnets (when using magnetic beads) and the like.

As used herein “isolating” refers to isolation of the composition comprising the rnicroalgae from the culture medium.

As used herein “harvesting” refers to isolating the microalgae from the composition i.e., disintegrating the compartmentalized structure.

It will be appreciated that the culture can be subjected to differential harvesting and processing, whereby for separate harvesting/isolation of microalgae from different compartments is performed such as based on different solubility of peripheral and central compartments (e.g. alginate vs chitosan) in various solvents.

An exemplary embodiment is provided below.

Calcium alginate is soluble in solutions of sodium polyphosphate and sodium carbonate with neutral pH, but chitosan is soluble in acidic solutions (pH<6).

In this case, exposure of compartmentalized capsules to salt solution with neutral pH will dissolve only peripheral compartment made from alginate. Microalgae (e.g. Spirulina) will be released in the solution and may be harvested using centrifugation. Chlorella will be released from alginate/chitosan capsules after exposure to acidic solution.

According to a specific embodiment, a concentration of the obligate photoautotrophic microalgae in the capsule is 10⁶-10¹⁰ cells/cm³ capsule.

According to a specific embodiment, a concentration of the obligate heterotrophic or mixotrophic microalgae in the capsule is 10⁶-10¹⁰ cells/cm³ capsule.

Microalgae can be used fresh or stored for variable time periods e.g., of at least but not limited to 3 months or at least 1 month at 4° C. in the dark and for extended time periods of at least, but not limited to over one year, with preference of about 3 months under conditions of light. Under conditions of darkness, the obligatory photoautotroph algae do not multiply, while exposure to light causes algae to photosynthesize and multiply.

The methods of microalgae entrapment allows to preserve the natural ingredients as the color of the algae, while the isolating capsules can be formed of different materials different shapes, size and colors. In addition, additives, such as food flavorings, aromas, food colorants, and preservatives can optionally be added.

The resultant products are used typically in the food, cosmetic or therapeutic industries, dependent on the type of microalgae employed.

For instance, in view of its high protein content, Spirulina may be a useful adjunct in the prevention and treatment of protein energy malnutrition (PEM) in children. Spirulina is rich in carotenoids with about 50% occurring as β-carotene, a principal provitamin A carotenoid. β-carotene in Spirulina, as in higher plants, is contained in chloroplasts and is associated with carotenoid binding proteins. However, due to its simple matrix (unicellular), it is thought to be more digestible than leafy green vegetables such as spinach. Adding Spirulina to meals has been reported to have favorable effects on glycemic control and lipid patterns, thus being of potential usefulness in the therapy of Diabetes mellitus Type II and in the control of cardiovascular risk factors. Spirulina also showed as an effective source to provide zeaxanthin, a component also found in human macular.

Chlorella contains large quantities of folate, vitamin B-12 and iron, and can help improve anemia and hypertensive disorders. Chlorella also contains Chlorella Growth Factor that can strengthen immunity and prevent or destroy cancer lesions. Chlorella food supplement products are able to enhance elimination of toxic heavy metals from organism.

Dunaliella is a genus of unicellular algae belonging to the family Polyblepharidaceae, that which lacks a rigid cell wall. This is a salt water alga and unlike spirulina and chlorella with high levels of proteins, it is very rich in mixed carotenes and xanthophylls (zeaxanthin, lutein, cryptoxanthin, violaxanthin, and echinenone). Dunaliella (Rhodophyta) provides the highest density of natural carotenoids of all the plants and algae. Thus, Dunaliella provitamin A carotenes (located in the chloroplast) exhibit bright red color. While the provitamin A carotenoids of Dunaliella are the highest among the three algae (as high as 13.8% of dry weight to be β-carotene) [34], the application of dunaliella has been focused in its pharmacological functions (antihypertensive, bronchodilator, etc.) and its use as natural food colorants or as an additive to cosmetics. Dunaliella carotene can protect cells against oxidant and photo damage.

Hence microalgae of the present inventions can be provided in the compositions described herein per se, mixed with other ingredients (therapeutic or food/feed where they are mixed with other nutritional, flavours, aromas and the like) or harvested following the methods described herein.

Example 1 of the examples section which follows provides a description of the method of some embodiments of the invention and should be acknowledged as part of the present specification.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Co-Encapsulation and Cultivation of Living Unicellular Mixotrophic Chlorella Algae and Obligatory Photoautotrophic Spirulina Algae

First Step—Production of Inner Compartments for Heterotrophic Growth of Chlorella Algae.

Living unicellular mixotrophic Chlorella algae are suspended in negatively charged salts of alginic acid (approximately 1/2, w/v), such as 0.5-10% sodium alginate. Drops of this mixture are dropped from about 2-30 cm into a setting bath containing fresh water (approximately 98%) and an edible water soluble calcium salt (approximately 2%), such as calcium chloride or calcium lactate. The drops (approximately 0.1 mm-3 mm) are left in the bath for a period of between about 1-30 minutes after which time the capsules become firm and are easily handled without breaking. Capsules are then removed by filtering from the bath and washed with water.

In some embodiments, the washed capsules may be incubated at room temperature in positively charged edible 0.5% solution of chitosan (pH˜6.0) or 1-10 min and washed with water. A thin layer of chitosan covered alginate capsules of Chlorella prevents leakage of proliferating Chlorella cells from alginate capsules, but allow diffusion of small soluble molecules (e.g. glucose, O₂, CO₂).

In some embodiments, alginate or/and chitosan may be mixed with non-transparent edible ingredients such as color particles, ink, etc. Such non-transparent matrix stimulates heterotrophic growth of Chlorella algae.

In some embodiments, alginate or/and chitosan may be mixed with floating particles made of edible oil or natural or artificial edible polymers, wax, air bubbles, aromatic oil, etc.

Second Step—Production of Compartmentalized Capsules Containing Heterotrophic and Photoautotrophic Algae Immobilized in Different Compartments

Living unicellular obligatory photoautotrophic Spirulina algae are suspended in transparent salts of alginic acid (approximately 1/2, w/v), such as 0.5-10% sodium alginate. Alginate capsules with Chlorella algae (first step) are transferred in alginate suspension of Spirulina algae. Drops of this mixture containing alginate/chitosan capsules of Chlorella and suspension of Spirulina algae in alginate are dropped from about 2-30 cm into a setting bath containing water (approximately 98%) and an edible water soluble calcium salt (approximately 2%), such as calcium chloride or calcium lactate. The drops (approximately 0.5 mm-20 mm) are left in the bath for a period of between about 1-30 minutes after which time the capsules become firm and are easily handled without breaking. Capsules are then removed from the bath and washed.

In some embodiments, suspension of Spirulina in alginate may be mixed with floating particles made of edible oil or natural or artificial edible polymers, wax, air bubbles, aromatic oil, etc. The floating particles lifting the algae containing capsules to water surface for better exposure to natural light in case of cultivation of algae-containing capsules in opened pond.

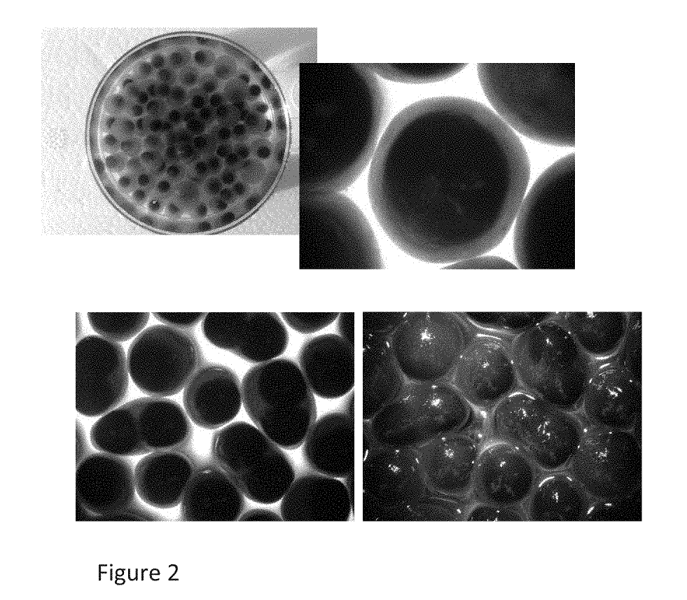

Images of capsules generated according to the present teachings are shown in FIG. 2.

Third Step—Cultivation of Algae Containing Capsules

Compartmentalized capsules containing heterotrophic and photoautotrophic algae are cultivated in medium such as water, containing various salts and a fixed carbon source (e.g. glucose). The medium contains NH4.NO3 (0.125 g/L), CaCl2.2H2O (0.025 g/L), MgSO4.7H2O (0.075 g/L), KQHPO4 (0.075 g/L), KHZPO4 (0.175 g/L), NaCl (0.025 g/L) and glucose (0.1 g/L-2 g/L) depending on Chlorella algae strain.

During cultivation, the immobilized microalgae are illuminated with visible or artificial light. The illumination activates photosynthesis of obligatory photoautotrophic algae (e.g. Spirulina) which release oxygen used by Chlorella for heterotrophic growth. At the same time, Chlorella algae release CO2 to be consumed by Spirulina for photosynthetic growth (mutual symbiosis between heterotrophic and photoautotrophic algae.

Fourth Step—Harvesting

1. Whole floating capsules may be harvested by simple methods using mesh etc.

2. Separate harvesting/isolation of microalgae from different compartments is based on different solubility of peripheral and central compartments (e.g. alginate vs chitosan) in various solvents.

For example:

Calcium alginate is soluble in solutions of sodium polyphosphate and sodium carbonate with neutral pH, but chitosan is soluble in acidic solutions (pH˜5) only.

In this case, exposure of compartmentalized capsules to salt solution with neutral pH will dissolve only peripheral compartment made from alginate. Microalgae (e.g. Spirulina) will be released in the solution and may be harvested using centrifugation. Chlorella will be released from alginate/chitosan capsules after exposure to acidic solution.

### Example 2

### Effect of Co-Encapsulation on Cell Viability

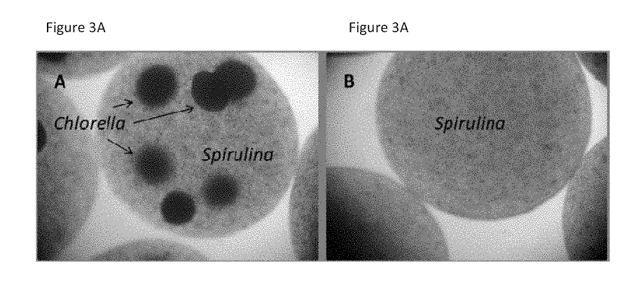

Co-encapsulation of photoautotrophic and mixotrophic micro-algae in compartmentalized alginate beads (as described in Example 1) offers also protection for one or both of the co-cultured microalgae species probably due to synergistic effects of antioxidant and antibacterial properties of the immobilized microalgae species. Images presented in FIGS. 3A-B demonstrate that co-encapsulation of photoautotrophic Spirulina and mixotrophic Chlorella micro-algae in compartmentalized alginate beads is able to prolong shelf life and preserve marketable characteristic of the developed product for several weeks during storage in darkness at room temperature. FIG. 3A shows the well preserved structure of Spirulina cells co-encapsulated with Chlorella algae in darkness at room temperature. In contrast, Spirulina micro-algae alone stored at the same conditions were bleached and disintegrated (FIG. 3B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. (canceled)

2. A compartmentalized composition comprising at least two compartments wherein a first compartment of said at least two compartments comprises an obligate photoautotrophic microalgae and a second compartment of said at least two compartments comprises an obligate heterotrophic or mixotrophic microalgae, said compartments are designed of a structure and/or composition ensuring symbiosis between said obligate photoautotrophic microalgae and said obligate heterotrophic or mixotrophic microalgae.

3. The compartmentalized composition of claim 2, wherein said first compartment of said at least two compartments is transparent to light and wherein when said second compartment comprises mixotrophic microalgae said second compartment is non-transparent to light.

4. The composition of claim 2, allowing free diffusion of small molecules, minerals and gas between said at least two compartments.

5-6. (canceled)

7. The composition of claim 2, wherein a concentration of said obligate photoautotrophic microalgae in said capsule is 10⁶-10¹⁰ cells/cm³ capsule or, wherein a concentration of said obligate heterotrophic or mixotrophic microalgae in said capsule is 10⁶-10¹⁰ cells/cm³ capsule.

8. (canceled)

9. The composition of claim 2, wherein said microalgae are viable.

10. The composition of claim 2, wherein said capsule is 0.1-20 mm in diameter.

11. (canceled)

12. The composition of claim 2, wherein said obligate photoautotrophic microalgae are present in said first compartment in at least 90% purity and said obligate heterotrophic or mixotrophic microalgae are present in said second compartment in at least 90% purity.

13. The composition of claim 2, further comprising a floating element rendering the composition floatable.

14-17. (canceled)

18. The composition of claim 2, wherein said first compartment encapsulates said second compartment.

19. The composition of claim 2, wherein said obligate photoautotrophic microalgae are selected from the group consisting of Dunaliella sp., Nannochloropsis sp., Synechococcus sp. and Spirulina sp.

20. The composition of claim 2, wherein said heterotrophic microalgae or said mixotrophic microalgae are characterized by growth rate faster than that of said obligate photoautotrophic microalgae.

21. The composition of claim 3, wherein said obligate heterotrophic microalgae are selected from the group consisting of Schizochytrium sp. and Crypthecodinium sp.

22. The composition of claim 3 wherein said mixotrophic microalgae are selected from the group consisting of Chlorella sp. and Chlamydomonas sp.

23. The composition of claim 2, wherein said mixotrophic microalgae are from the group of Chlorella sp. and said obligate photoautotrophic microalgae are from the group of Spirulina sp.

24-26. (canceled)

27. The composition of claim 2, wherein said obligate photoautotrophic microalgae are encapsulated by a polymeric material.

28. The composition of claim 2, wherein said first compartment and said second compartment are composed of polymeric materials.

29. The composition of claim 27, wherein said polymeric material is light transparent.

30. The composition of claim 29, wherein said polymeric material is selected from the group consisting of alginate, agarose, gelatin and chitosan.

31. (canceled)

32. A method of producing a nutritional composition, the method comprising:
(a) producing a compartmentalized composition comprising at least two compartments wherein a first compartment of said at least two compartments comprises an obligate photoautotrophic microalgae and a second compartment of said at least two compartments comprises an obligate heterotrophic or mixotrophic microalgae, said compartments are designed of a structure and/or composition ensuring symbiosis between said obligate photoautotrophic microalgae and said obligate heterotrophic or mixotrophic microalgae; and
(b) culturing said microalgae in said particles, thereby producing said nutritional composition.

33-53. (canceled)
